(19) United States
(12) Patent Application Publication
(10) Pub. No.: US 2025/0199096 A1
(43) Pub. Date: Jun. 19, 2025

(54) MAGNETIC FIELD MEASUREMENT DEVICE FOR THE DETECTION OR IMAGING OF MAGNETIC PARTICLES

(71) Applicant: ASELSAN ELEKTRONIK SANAYI VE TICARET ANONIM SIRKETI, Ankara (TR)

(72) Inventor: Can Baris TOP, Ankara (TR)

(73) Assignee: ASELSAN ELEKTRONIK SANAYI VE TICARET ANONIM SIRKETI, Ankara (TR)

(21) Appl. No.: 18/847,312

(22) PCT Filed: Nov. 28, 2023
(86) PCT No.: PCT/TR2023/051415

§ 371 Date: Sep. 16, 2024

(30) Foreign Application Priority Data
Nov. 30, 2022 (TR) 2022/018221

(51) Int. Cl. G01R 33/26; G01R 33/12

(52) U.S. Cl. CPC G01R 33/26; G01R 33/1276

(57) ABSTRACT

A magnetic field measuring device for detection or imaging of magnetic nanoparticles is provided. The magnetic field measuring device includes at least one transmitting coil configured to generate a first magnetic field magnetizing the magnetic particles substantially uniformly in a sensing region to be measured, and zero magnetic field lines outside the sensing region, at least one coil drive and control circuit configured to drive at least one transmitting coil, at least one semiconductor material having at least one defect center, at least one optical assembly having at least one optical sensor configured to optically excite the defect center(s) and to detect the radiation produced by the defect center(s), at least one microwave antenna configured to excite the electron spin state of the defect center(s), and at least one microwave drive and control circuit configured to drive the microwave antenna.

### CROSS REFERENCE TO THE RELATED APPLICATIONS

This application is the national phase entry of International Application No. PCT/TR2023/051415, filed on Nov. 28, 2023, which is based upon and claims priority to Turkish Patent Application No. 2022/018221, filed on Nov. 30, 2022, the entire contents of which are incorporated herein by reference.

### TECHNICAL FIELD

The present invention relates to a magnetic field measurement device for detection or imaging of magnetic nanoparticles.

### BACKGROUND

Magnetic particles are particles in which a magnetic core, such as iron oxide, is coated for the purposes of biocompatibility, medical functionality, increased residence time in the body, and prevention of coalescence. The use of magnetic particles in medical imaging and therapy applications for various purposes has been proposed in the known state of the art. Magnetic particle detection and magnetic particle imaging methods are used to detect or imaging the localization and amount of magnetic particles in the body.

In magnetic particle detection or imaging methods, magnetic nanoparticles or cells labeled with magnetic nanoparticles are introduced into the body and the response of the particles inside the body to the externally applied time-varying magnetic field is measured. Using this measured response, the positions and densities of the magnetic nanoparticles can be detected or visualized. This information can be used for both diagnosis and treatment.

In magnetic particle sensing, magnetic particles pre-delivered to a sensing site (e.g. inside the body) are detected by manual scan path by means of a magnetic field probe outside the body. In the U.S. Pat. No. 9,523,748 in the known state of the art, an assembly for such a method is proposed. In magnetic particle detection systems, a time-varying magnetic field is created using a transmitting coil. The magnetization response of magnetic particles to the magnetic field generated by the transmitting coil is detected using receiving coils. A probe containing the transmitting and receiving coils can be scanned manually outside the body to localize the area where the magnetic particles are located.

In magnetic particle imaging methods, images of the distribution of magnetic particles in the body can be obtained, as described in the U.S. Pat. No. 7,370,656 in the known state of the art. The magnetic particle imaging method is based on the rapid and non-linear magnetization of magnetic nanoparticles. For imaging, an inhomogeneous magnetic field that contains a point (magnetic field-free point-MFP) or line (zero magnetic field line-MFL) where the magnetic field is zero, is first generated. The region containing the point or line where the magnetic field is zero can also be referred to as the magnetic field-free region. A magnetic field containing a magnetic field-free region is referred to as a selection field, because its distribution selects the imaged region. Magnetic nanoparticles inside the magnetic field-free region can be magnetized by a magnetic field other than the selection field, while magnetic nanoparticles outside the magnetic field-free region cannot react to an external magnetic field as they are magnetically saturated. In magnetic particle imaging method, in addition to the selection field, another time-varying magnetic field, also known as a dynamic magnetic field or a driving magnetic field, is applied. This dynamic magnetic field excites the magnetic nanoparticles in the magnetic field-free region, causing the magnetization of the magnetic nanoparticles to change dynamically. This time-varying magnetization is detected by a magnetic receiver. The detected magnetization originates from the magnetic nanoparticles that are present only in the magnetic field-free region and increases proportionally to the magnetic nanoparticle density. The magnetic nanoparticle distribution in the tissue is obtained by scanning the magnetic field-free region in the tissue and processing the signals received from the magnetic nanoparticles. As the amplitude of the dynamic magnetic field increases, the imaging area expands. However, since heating and nerve stimulation can occur especially at high frequency, the frequency and amplitude of the dynamic magnetic field should be limited patient safety. With a dynamic magnetic field used within safety limits, the imaging area is centimeters in size. In order to scan a larger imaging area, a third magnetic field with a low frequency and which changes the focus of the scan point is used. When said third magnetic field is added to the selection field, it is characterized as a focal field because it displaces a magnetic field-free region. As a result, in order to collect data and perform imaging from an imaging area that can be used in the clinic in the magnetic particle imaging method, it is necessary to generate three separate magnetic fields including a selection field that does not change with time, a focus field that changes slowly with time and a dynamic field that changes rapidly with time.

The dynamic field used in the magnetic particle imaging method and the time-varying magnetic field used in magnetic particle detection are generated by the transmitting electromagnetic coils. The response of magnetic particles to the variable magnetic field is usually measured by magnetic induction using receiving coils as described in the U.S. Pat. Nos. 20,192,23975 and 8,183,861 and European patent document EP3378389 in the known state of the art.

During the imaging or detection of magnetic particles, the excitation of magnetic particles using transmitting coils and the reception of signals from the receiving coils are performed simultaneously. Therefore, not only the magnetization of magnetic particles but also the magnetic field generated by the transmitting coil induces voltage in the receiving coils. Since the voltage induced by the transmitting coil is much higher than the voltage induced by the magnetic particle magnetization, it is necessary to separate the magnetic particle magnetization from the effect of the transmitting magnetic field. In the known state of the art, filtering, gradiometric receiving coils and conjugate receiving system methods are used for this purpose.

The filtering method takes advantage of the fact that the magnetization signal of magnetic nanoparticles is non-linear and contains high harmonic frequency components. The first harmonic of the magnetic particle-induced signal is at the same frequency as the transmitting coil signal. The signal induced in the receiving coil is passed through a band-stop filter to suppress the first harmonic of the received signal as much as possible. This eliminates the transmitting coil signal (Bente, Klaas, et al. “Electronic field free line rotation and relaxation deconvolution in magnetic particle imaging.” IEEE Transactions on Medical Imaging 34.2 (2014): 644-651). However, since this method eliminates the signal of the transmitting coil while also eliminating the magnetization signal of the magnetic particles, a large portion of the magnetic particle signal is lost, reducing the signal-to-noise ratio.

In the method using gradiometric receiving coils, two conjugate receiving coils with opposite polarity with respect to each other are used. These coils are placed in such a way as to zero the voltage induced by the transmitting coil signal (Can Baris Top and Alper Gungor. “Tomographic field free line magnetic particle imaging with an open-sided scanner configuration.” IEEE Transactions on Medical Imaging 39.12 (2020): 4164-4173). Since the magnetic particles must induce voltage in only one of the receiving coils to receive the magnetic particle signal, and therefore the other receiving coils must be located in a region where the particles will not be magnetized, the field of view of the imaging system is limited when using said method.

The conjugate receive system method also works in a similar way to the method using gradiometric receiving coils. In the conjugate receive system method, a replica of the transmitting and receiving coil structure is added to the system to obtain the signal to be received in the absence of magnetic particles (Mattingly, Eli, et al. “Drive and receive coil design for a human-scale MPI system.” International Journal on Magnetic Particle Imaging 8.1 Suppl 1 (2022)). The signal due to magnetic particle magnetization is separated by subtracting this signal from the signal from the main system. In contrast, since the conjugate receive system method requires two separate transmitting and receiving systems, it increases the cost and power consumption, and at the same time there is a risk of distortions in the received signal due to differences in the main system and the conjugate system such as ambient temperature.

Therefore, in the known state of the art, a device is needed that greatly reduces the influence of the magnetic field generated by the transmitting coil on the sensors measuring the magnetic particle response.

As mentioned earlier, in magnetic particle imaging and sensing, the magnetization signal of magnetic particles is usually received by the voltage induced in the receiving coils. The voltage induced in a coil is directly proportional to the time-dependent derivative of the magnetic flux passing through the coil. The magnetization of magnetic nanoparticles varies depending on the magnetic field emitted from the transmitting coil. Therefore, the amplitude of the signal measured by the receiving coil varies depending on the frequency of the magnetic field emitted from the transmitting coil, and the received signal level decreases as the frequency decreases. The frequency used for magnetic particle imaging is usually around 25 kHz. It has been shown that at frequencies below this value the signal level drops significantly (Top, C. B. “An arbitrary waveform magnetic nanoparticle relaxometer with an asymmetrical three-section gradiometric receive coil.” Turkish journal of electrical engineering and computer sciences 28.3 (2020): 1344-1354). On the other hand, as the diameter of the magnetic particle increases, the magnetization curve steepens and accordingly the imaging resolution increases. However, magnetic particles with relatively large diameters cannot respond to high frequencies and therefore cannot be detected by inductive receivers. This limits the resolution of magnetic particle imaging.

Therefore, in the known state of the art, there is a need for a device that allows the magnetization response of magnetic particles to be measured at low excitation frequencies in order to allow relatively large magnetic particles to respond and to increase image resolution. In addition, since the temperature and viscosity dependent variations of magnetic particles have a distinctive characteristic at relatively low frequencies, in the known state of the art there is also a need for a device that allows the measurement of the magnetic particle magnetization variation with respect to the temperature and viscosity of the medium.

In addition to all these, in the known state of the art, the use of non-inductive atomic magnetometers for magnetic particle imaging, which detect a signal directly related to particle magnetization is also proposed (Colombo, Simone, et al. “Imaging magnetic nanoparticle distributions by atomic magnetometry-based susceptometry.” IEEE Transactions on Medical Imaging 39.4 (2019): 922-933). In this proposed method, an optically pumped magnetometer is placed outside the transmitting coils and the magnetic particle-induced magnetization signal is obtained by this said magnetometer. In order to reduce the magnetic field generated by the transmitting coils, reverse polarity compensation coils are used, which are arranged outside the transmitting coils and generate a magnetic field in the opposite direction with respect to the transmitting coil in the region where the particles are located. Therefore, much more current is supplied to the transmitting coil to apply the desired magnetic field to the magnetic particles compared to the case without compensation coils. This both reduces system efficiency and increases system cost and power consumption as higher current generators have to be used. Since said compensation coils reduce the magnetic field outside of the imaging field, the atomic magnetometer has to be placed far away from the imaging field. Since the magnetic field decreases with the cube of the distance, such a distant placement of the atomic magnetometer results in a significant decrease in the received signal level and signal-to-noise ratio.

In an embodiment of the known state of the art enabling magnetic particle detection, an approach based on optical detection and magnetic resonance signal receive method is applied (Kuwahata, A., Kitaizumi, T., Saichi, K. et al. Magnetometer with nitrogen-vacancy center in a bulk diamond for detecting magnetic nanoparticles in biomedical applications. Sci Rep 10, 2483 (2020)). In said method, the compensation coils described above are also used. Since the compensation coils must be of opposite polarity to the transmitting coils, the problems mentioned above also apply to this embodiment.

Therefore, in the known state of the art, there is a need for a device which eliminates the need for a compensation coil altogether or, where a compensation coil is used, ensures that the magnetic field generated by the compensation coils in the region of the magnetic particles is negligibly smaller than the magnetic field generated by the transmitting coils in the region of the magnetic particles.

### SUMMARY

The purpose of the present invention is to provide a high-precision magnetic field measurement device that can efficiently measure the magnetization response of magnetic particles in a sensing region, especially at excitation frequencies higher than 5 kHz, but also at low excitation frequencies such as 0-5 kHz.

A magnetic field measuring device as defined in the first claim and the dependent claims correspondingly to this claim realized to achieve the purpose of the present invention, which provides high sensitivity detection of a magnetic particle signal for detecting or imaging magnetic particles; at least one transmitting coil configured to generate a first magnetic field magnetizing the magnetic particles substantially uniformly in a sensing region to be measured containing a plurality of magnetic particles and lines on the axis of extension where the magnetic field is zeroed, at least one coil drive and control circuit configured to drive the transmitting coil in a controlled manner, at least one semiconductor material comprising at least one defect center whose electron spin and energy state can be changed when excited with electromagnetic energy, at least one optical assembly having at least one optical sensor configured to optically excite the defect centers and to detect radiation emitted from the defect centers due to such optical excitation, at least one microwave antenna configured to excite the electron spin state of the defect centers, and at least one microwave drive and control circuit configured to operate the microwave antenna in a controlled manner, wherein the semiconductor material in the inventive magnetic field measuring device is positioned such that the defect centers are located substantially above at least one line where the magnetic field is zero.

In a preferred embodiment of the invention, the magnetization response of magnetic nanoparticles is detected by magnetic field sensors based on optically detected magnetic resonance (ODMR) measurement. This type of sensor is able to measure the magnetization directly and not the derivative of the magnetic flux as in inductive sensing. This eliminates the limitations of in the known state of for low-frequency excitation and large magnetic particle diameter. Furthermore, the development of ODMR sensors, which have higher sensitivity than inductive coils, can improve the sensitivity of magnetic particle detection and imaging. For ODMR, nitrogen-vacancy defect centers, which are naturally found in diamonds or synthetically obtainable, are preferably used. In different embodiments, the negatively charged boron-vacancy defect center in the hexagonal boron nitrate structure or the single carbon defect center or the double-vacancy defect center in the silicon carbide structure can be used. The magnetic field-free region generated by the transmitting coil elements can be linear, so that optically sensed magnetic resonance sensors can be positioned in a linear array. In this way, the sensor coverage areas and signal-to-noise ratio can be increased. It is also possible to apply Ramsey, Pulsed ODMR or Spin echo ODMR methods using pulsed signals in the known state of the art to increase measurement sensitivity.

In one embodiment, the magnetization signal due to the relaxation response of the magnetic particles after the excitation of the transmitting coils is terminated is measured by the ODMR method. In this case, since the magnetic field sensor is not affected by the magnetic field emitted by the transmitting coil element before relaxation, the response can be measured precisely.

Especially since the defect centers are directional, it is possible to measure the vector magnetic field. The negatively charged nitrogen-vacancy defect centers in diamond can be four-directional due to the structure of the atomic bonds. A single negatively charged nitrogen defect center can be used for magnetic field measurement, or a diamond with many negatively charged nitrogen defect centers can be also used.

In magnetic particle sensing or imaging, the direction of the magnetic field generated by the magnetic nanoparticles is determined by the direction of the magnetic field generated by the transmitting coil. In one embodiment, the angle of the diamond containing a single or plurality of negatively charged nitrogen-vacancy defect centers is adjusted to maximally detect the signal in the magnetic particle magnetization direction.

Since the energy level of the negatively charged nitrogen-vacancy defect centers in diamond between the mₛ=+1 and −1 electron spin states is equal to the projection of the magnetic field in the direction of the negatively charged nitrogen-vacancy defect centers, it is possible to measure the magnetic particle magnetization vectorially. In one embodiment, the vectorial variation of magnetic particle magnetization is measured by following the resonance frequencies of negatively charged nitrogen defect centers in four different directions in the ODMR spectrum. Magnetic particles react to the magnetic field by two different mechanisms, Neel and Brownian: In the Neel mechanism, the particles do not physically rotate and the magnetization changes rapidly, while in the Brownian mechanism, the particles physically rotate. By measuring the vector magnetic field, it is possible to distinguish between these two mechanisms. By measuring the magnetic particle magnetization vectorially, the magnetization change mechanics of the particles can be obtained and information about the ambient viscosity and temperature can be obtained.

### BRIEF DESCRIPTION OF THE DRAWINGS

The magnetic field measurement device for achieving the purpose of the present invention is shown in the accompanying figures;

FIG. 1-A representative schematic view of an embodiment of the inventive magnetic field measuring device.

FIG. 2-A simplified representative view of the magnetic field distribution generated by the transmitting coils in one embodiment of the inventive magnetic field measuring device.

FIG. 3-A representative view of a diamond crystal containing a negatively charged nitrogen-vacancy defect center in its structure in one embodiment of the inventive magnetic field measurement device.

FIG. 4-A representative view of the optical excitation and optical emission in the energy diagram of negatively charged nitrogen-vacancy defect centers in diamond in one embodiment of the inventive magnetic field measuring device.

FIG. 5-A representative view of the optical excitation and optical emission of the energy diagram of negatively charged nitrogen-vacancy defect centers in diamond in a microwave-excited state in one embodiment of the inventive magnetic field measuring device.

FIG. 6A-A drawing representing the magnetic field around the line where the magnetic field generated by the transmitting coils is zero in an embodiment of the inventive magnetic field measuring device.

FIG. 6B-A drawing representing a representative representation of an embodiment of the inventive magnetic field measuring device, wherein the position of the line at which the magnetic field generated by the transmitting coils is zero is changed by the magnetic field generated by the compensation coils.

FIG. 6C-A drawing representing a representative representation of the first magnetic field generated by the transmitting coils, the magnetic field generated by the compensation coils and the total magnetic field around the line where the magnetic field generated by the transmitting coils is zero in an embodiment of the inventive magnetic field measuring device.

FIG. 7-A representative schematic view showing the placement of the compensation coils in an embodiment of the inventive magnetic field measuring device.

FIG. 8-A representative schematic view showing the placement of the compensation coils in another embodiment of the inventive magnetic field measuring device.

The parts in the figures are individually numbered and the equivalents of these numbers are given below.
1. Magnetic field measuring device
2. Transmitting coil
3. Coil drive and control circuit
4. Semiconductor material
5. Optical assembly
6. Microwave antenna
7. Microwave drive and control circuit
8. Compensation coil
SR. Sensing region
MF1. First magnetic field
MFL. Zero Magnetic field line
DC. Defect center
mₛ. Electron spin state of the defect center
MF2. Second magnetic field
³A. Base state energy level
³E. Excited state energy level

### DETAILED DESCRIPTION OF THE EMBODIMENTS

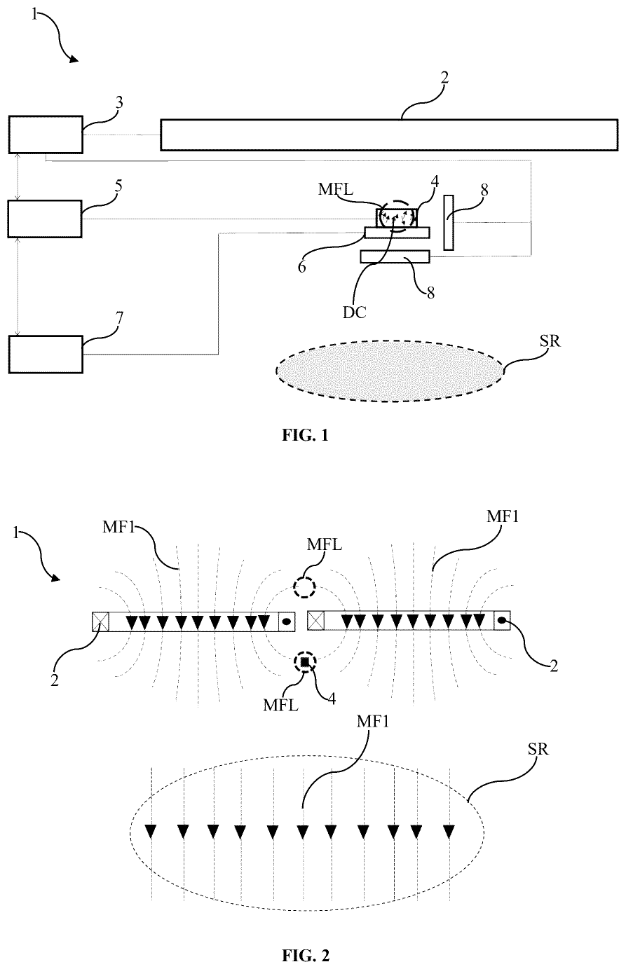

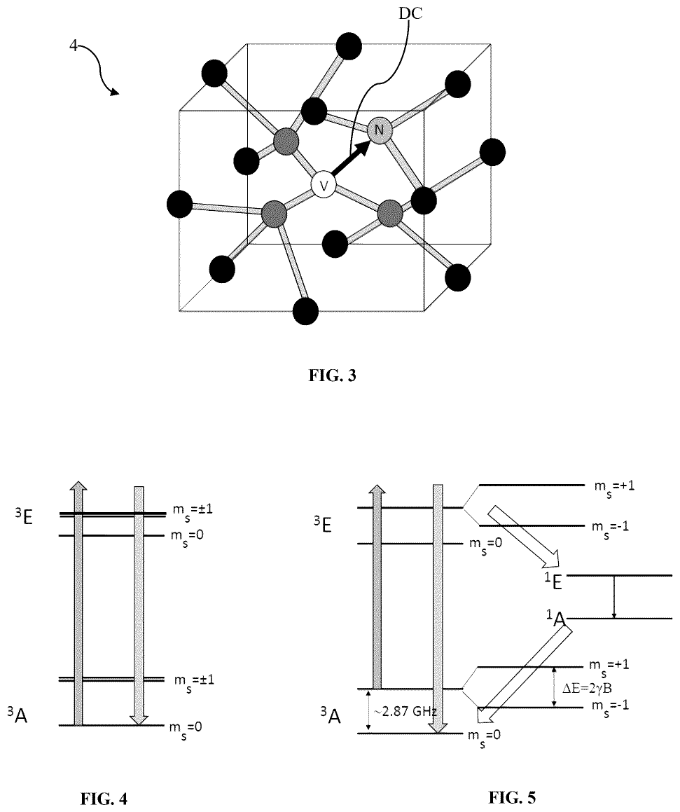

A magnetic field measuring device (1) for high sensitivity detection of a magnetic particle signal in the detection or imaging of magnetic particles; at least one at least one transmitting coil (2) configured to generate a first magnetic field (MF1) magnetizing the magnetic particles substantially coherently in a sensing region (SR) to be measured containing a plurality of magnetic particles, and zero magnetic field lines (MFL) outside sensing region (SR), at least one coil drive and control circuit (3) configured to drive at least one transmitting coil (2), at least one semiconductor material (4) having at least one defect center (DC) whose electron spin and energy state can be changed when excited by electromagnetic energy, at least one optical assembly (5) having at least one optical sensor (not shown) configured to optically excite a defect center (DC) in the semiconductor material (4) and to detect the radiation emitted by the defect center (DC) in response to the optical excitation, at least one microwave antenna (6) configured to excite the electron spin state of the defect center (DC) located within the semiconductor material (4) and at least one microwave drive and control circuit (7) configured to drive the microwave antenna (6) (FIG. 1). In a preferred embodiment, the magnetic field measuring device (1) comprises a plurality of transmitting coils (2), preferably two. In one embodiment, the transmitting coils (2) are adapted as rectangular electromagnets arranged side by side on the same plane and extending substantially parallel to each other, preferably having substantially the same size, polarization and number of turns. When the transmitting coils (2) are fed with the same current, two lines (MFL) are formed on which the magnetic field is zero, taking the midpoint of the edges lying parallel to each other as the center and extending parallel to the edges lying parallel to each other on an axis perpendicular to the plane in which the transmitting coils (2) extend. Since the position of the lines (MFL) on which the magnetic field is zero depends on the center positions of the transmitting coils (2) and the distance between them, this position can be adjusted according to the specific embodiment. Depending on the requirements in different embodiments of the invention, by adjusting this position, the zero magnetic field lines (MFL) can be moved closer to or further away from the transmitting coils (2). In different embodiments, the transmitting coils (2) can also be designed to be intertwined with each other. By applying electromagnetic energy at microwave frequency to the defect centers (DC) in the semiconductor material (4) through the microwave antenna (6), the magnetic field sensitive resonance effects can be physically measured by exciting the electron spin states from the mₛ=0 state to the mₛ=+1 and mₛ=−1 states. The semiconductor material (4) preferably comprises at least one diamond, which already contains negatively charged nitrogen-vacancy defect centers (DC). In alternative embodiments, the semiconductor material (4) may comprise, without limitation, hexagonal boron nitrate having a negatively charged boron-vacancy defect center (DC) or a single carbon defect center (DC), or silicon carbide having a double-vacancy defect center (DC). In the following detailed description, reference will be made to the negatively charged nitrogen-vacancy defect centers (DC) in the structure of diamond, and this does not mean that the invention is limited to negatively charged nitrogen-vacancy defect centers (DC). In the measurements carried out by means of the magnetic field measuring device (1), the negatively charged nitrogen-vacancy defect centers (DC) in the base energy state (3A) with zero electron spin state (mₛ=0) are optically excited by means of the optical assembly (5). The energy state of the optically excited negatively charged nitrogen-vacancy defect centers (DC) changes to the excited state (³E), but when the optical excitation is stopped, the already excited negatively charged nitrogen-vacancy defect centers (DC) return to their unexcited state, i.e. their base energy states (³A). During this return, the negatively charged nitrogen-vacancy defect centers (DC) emit photons at the optical frequency (FIG. 4). When the negatively charged nitrogen-vacancy defect centers (DC) in the base energy state (³A) are energized at a specific microwave frequency around 2.87 GHz by means of the microwave antenna (6), it is possible to increase the electron spin state (from mₛ=0 to mₛ=±1) (FIG. 5). Negatively charged nitrogen-vacancy defect centers (DCs), whose electron spin state is elevated (mₛ=±1) in the base energy state (³A), are again elevated to the excited state (³E) when optically excited by means of the optical assembly (5). When the excitation is terminated, the negatively charged nitrogen-vacancy defect centers (DCs) can return to the base energy state with zero electron spin state (mₛ=0) in two different ways, either directly or by passing through intermediate singlet energy steps (FIG. 5). In the direct transition, there is photon emission from the negatively charged nitrogen-vacancy defect centers (DCs), while there is no photon emission in the intermediate step transition. By measuring the amount of light emitted from the negatively charged nitrogen-vacancy defect centers (DCs) at these stages, it can be understood whether the electron spin states (mₛ=±1) of the negatively charged nitrogen-vacancy defect centers (DCs) transition to the elevated state. In the presence of a magnetic field in the medium, the raised electron spin state (mₛ=±1) is split into two (mₛ=+1 and mₛ=−1) by the Zeeman effect. The energy between these two raised electron spin states (mₛ=+1 and mₛ=−1) is ΔE−2γB, where β˜28 [GHz/T] is the gyromagnetic ratio and B [T] is the ambient magnetic field intensity. Due to this energy dissociation, the microwave resonant frequency, which allows the transition from the zero electron spin state (mₛ=0) to different elevated electron spin states (mₛ=+1 or mₛ=−1), also changes. In order to perform magnetic field measurement in magnetic imaging or detection by means of the magnetic field measuring device (1), the negatively charged nitrogen-vacancy defect centers (DC) in the base energy state (³A) are first excited to the excited energy level (³E) by means of the optical assembly (5), preferably with light having a wavelength of about 530 nm. In one embodiment, the light is transported from the optical assembly (5) to the semiconductor material (4) via a fiber optic cable. Following optical excitation of the negatively charged nitrogen-vacancy defect centers (DC), a microwave signal is applied to the negatively charged nitrogen-vacancy defect centers (DC) by means of a microwave antenna (6) under the control of a microwave drive and control circuit (7), and the amount of light emitted from the negatively charged nitrogen-vacancy defect centers (DC) is measured by an optical sensor in the optical assembly (5). In one embodiment, the light emitted from the negatively charged nitrogen-vacancy defect centers (DC) is transported to the optical sensor via a fiber optic cable. After the light is received by the optical sensor, the microwave frequency is scanned by the microwave drive and control circuit (7) to obtain information on the frequencies at which the amount of light decreases, and using this information, the total magnetic field in the environment, including the magnetization of magnetic particles, is calculated.

In the inventive magnetic field measuring device (1), the semiconductor material (4) is arranged so that the negatively charged nitrogen-vacancy defect center (DC) in its content coincides with at least one line (MFL) where the magnetic field is zero. By placing the semiconductor material (4), which is configured to detect the magnetization of magnetic particles in the sensing region (SR), on the zero magnetic field line (MFL), the first magnetic fields (MF1) generated by the transmitting coils (2) are not detected by the negatively charged nitrogen-vacancy defect center(s) (DC) in the semiconductor material (4), or are detected at a negligible level, and only the magnetization of magnetic particles in the sensing region (SR) is detected. This significantly increases the detection sensitivity of the inventive magnetic field measurement device (1).

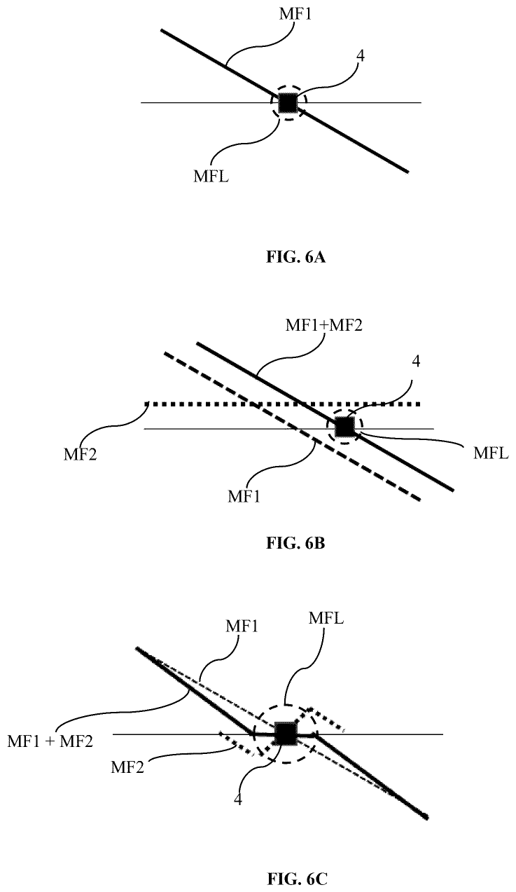

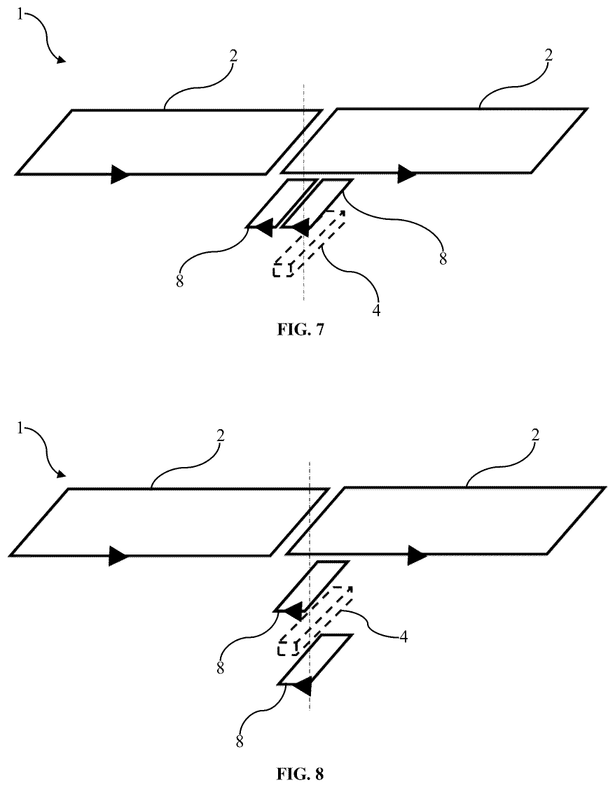

In one embodiment of the invention, the magnetic field measuring device (1) comprises at least one compensation coil (8) configured to generate at least a second magnetic field (MF2) for adjusting the position of the zero magnetic field lines (MFL) by moving the zero magnetic field lines (MFL) generated by the transmitting coils (2). In one embodiment, the magnetic field measuring device (1) comprises at least two compensation coils (8) configured to generate two different second magnetic fields (MF2) for moving the zero magnetic field line (MFL) in two different axes, one in the axis of extension of the zero magnetic field line (MFL) of the transmitting coils (2) and the other in the axis of extension of the zero magnetic field line (MFL) between the transmitting coils (2) parallel to each other (FIG. 1). The compensation coil (8) is driven by the coil drive and control circuit (3), preferably at the same frequency as the transmitting coils (2), either in phase or in antiphase. The second magnetic field (MF2) generated by the compensation coil (8) increases the detection sensitivity by ensuring that the positions of the negatively charged nitrogen-vacancy defect center(s) (DC) of the semiconductor material (4) are on the zero magnetic field lines (MFL) before the magnetic field measurement (FIG. 6B). Since the negatively charged nitrogen-vacancy defect centers (DCs) are already located almost in the region of the zero magnetic field lines (MFLs), the compensation coil (8) is fed with very small currents compared to the transmitting coils (2) to ensure precise positioning. Therefore, the second magnetic field (MF2) generated by the compensation coils (8) has no significant effect on the first magnetic field (MF1) generated by the transmitting coils (2) in the sensing region (SR) where magnetic particles are present.

In one embodiment of the invention, the compensation coil (8) is configured to generate a second magnetic field (MF2) having a magnetic field gradient which eliminates the variation (gradient) with position of the first magnetic field (MF1) generated by the transmitting coils (2) around the zero magnetic field line (MFL) (FIG. 6C). By this way, the volume of the magnetic field-free region containing zero magnetic field lines (MFL) is increased. In one embodiment, at least two compensation coils (8) arranged side by side with each other to generate a magnetic field gradient such that the first magnetic field (MF1) is zeroed with position (FIG. 7). In an alternative embodiment, at least two compensation coils (8) arranged parallel to each other, facing each other, are used to generate a magnetic field gradient such that the first magnetic field (MF1) is zeroed with position, and a second magnetic field (MF2) is generated in the opposite direction (FIG. 8). In these embodiments, the compensation coils (8) are arranged to form a magnetic field-free region in a region encompassing the zero magnetic field lines (MFL) generated by the transmitting coils (2). The transmitting coils (2) and the compensation coils (8) are fed in reverse phase to extend the zero magnetic field in the region where the negatively charged nitrogen-vacancy defect centers (DC) are located and which covers the zero magnetic field lines (MFL). For this to occur, the compensation coil (8) currents are regulated to generate a magnetic field gradient of the same amplitude as the magnetic field gradient generated by the transmitting coil (2) in the vicinity of the zero magnetic field lines (MFL), but in reverse phase. In one embodiment, the compensation coil (8) currents are adjusted using magnetic field measurement information measured by the inventive magnetic field measuring device (1). The compensation coil (8) currents that minimize the measured magnetic field can be found using various optimization and control algorithms known in the art. In some embodiments, the compensation coil (8) current may need to be changed during operation due to changes in ambient parameters such as temperature. In such a case, using feedback and optimization algorithms known in the art, the current of the compensation coil (8) is optimized to minimize the magnetic field measured by the magnetic field measuring device (1).

In one embodiment, the magnetization of magnetic particles is manipulated by applying a time-varying current, preferably sinusoidal, to the transmitting coils (2) for the detection of magnetic particles. For the detection of magnetic particles, an optical signal, preferably with a wavelength of about 530 nm, is sent to the negatively charged nitrogen-vacancy defect centers (DC). A microwave signal is also applied to the negatively charged nitrogen-vacancy defect centers (DC) by means of a microwave antenna (6) under the control of a microwave drive and control circuit (7). The microwave signal can be applied as a continuous wave or frequency modulated to increase detection sensitivity. The center frequency of the microwave signal applied to the negatively charged nitrogen-vacancy defect centers (DC) is scanned in a certain bandwidth. Meanwhile, the radiation emitted from the negatively charged nitrogen-vacancy defect centers (DC) is measured by the optical sensor in the optical assembly (5). In one embodiment, a “lock-in” amplifier is used to amplify the optical sensor signal. The lock-in amplifier amplifies the optical sensor signal with reference to the microwave signal applied to the negatively charged nitrogen-vacancy defect centers (DC). By detecting the resonant frequencies in the obtained microwave frequency-dependent optical radiation spectrum data, the magnetic field change due to the magnetization of magnetic particles on the negatively charged nitrogen-vacancy defect centers (DC) is detected. In one embodiment of the invention, the resonant frequency in the optical radiation spectrum is shifted by 28 Hz/nT.

In one embodiment of the invention, the coil drive and control circuit (3), the optical assembly (5) and the microwave drive and control circuit (7) are configured to communicate with each other in such a way as to exchange data in order to apply the signal waveforms required for magnetic field measurement to the transmitting coils (2), the compensation coils (8), the negatively charged nitrogen-vacancy defect centers (DC) and the microwave antenna (6). An additional control computer or circuit (not shown in the figures) can be used in alternative embodiments to optimize the synchronization and measurement of these signals.

In one embodiment of the invention, the microwave drive and control circuit (7) is configured to control the microwave antenna (6) to emit a microwave signal modulated at a frequency that excites “hyperfine” electron spin resonances to negatively charged nitrogen-vacancy defect centers (DC). This modulation improves the measurement sensitivity. The microwave drive and control circuit (7) controls the microwave antenna such that a microwave signal, preferably modulated at a frequency of 2.16 MHz, is emitted. In one embodiment of the invention, resonant frequencies are detected in the absence of magnetic particles and the frequency point with the highest sensitivity (the frequency point at which the variation of optical radiation with frequency is maximum) is determined. In this embodiment, microwave antenna (6) applies microwave at this center frequency point. In the presence of magnetic particles, the magnetization of magnetic particles is measured by measuring the deviation from this center frequency point.

In one embodiment of the invention, a plurality of transmitting coils (2) are positioned at different positions around the sensing region (SR) in such a way as to increase the intensity of the first magnetic field (MF1) in the sensing region (SR) or to homogenize the first magnetic field (MF1) in the sensing region (SR). In this way, magnetic field measurement can be performed more precisely.

In one embodiment of the invention, the magnetic field measuring device (1) further comprises at least one permanent magnet (not shown) arranged to separate the directions of the negatively charged nitrogen-vacancy defect centers (DC). According to the amplitude of the projection of the magnetic field direction generated by this magnet in the direction of the negatively charged nitrogen-vacancy defect centers (DC) in the semiconductor material (4), the separation of negatively charged nitrogen-vacancy defect centers (DC) in different directions in the optically sensed magnetic resonance spectrum is provided. In magnetic particle imaging, this separation occurs spontaneously due to the presence of a static selection field and slowly time-varying focal fields.

The present invention also relates to a magnetic particle detection system comprising a magnetic field measuring device (1) of the type described above.

The present invention also relates to a magnetic particle imaging system comprising a magnetic field measuring device (1) of the type described above.

The present invention also relates to a magnetic particle relaxometer system comprising a magnetic field measuring device (1) of the type described above.

In the inventive magnetic field measurement device (1), by arranging the defect center(s) (DC) of the semiconductor material (4) in such a way that the defect center(s) (DC) in the semiconductor material (4) correspond to at least one line (MFL) where the magnetic field is zero, the first magnetic fields (MF1) generated by the transmitting coils (2) are not detected through the defect center(s) (DC) of the semiconductor material (4) or are detected at a negligible level, and only the magnetic particle magnetization in the sensing region (SR) is detected. This significantly increases the detection sensitivity of the inventive magnetic field measurement device (1).

What is claimed is:

1. A magnetic field measuring device, that-wherein the magnetic field measuring device provides a high sensitivity detection of a magnetic particle signal in a detection or imaging of magnetic particles and comprises:
at least one transmitting coil configured to generate a first magnetic field, wherein the first magnetic field magnetizes the magnetic particles nearly coherently in a sensing region to be measured containing a plurality of magnetic particles, and zero magnetic field lines outside the sensing region to be measured,
at least one coil drive and control circuit configured to drive the at least one transmitting coil,
at least one semiconductor material having at least one defect center,
at least one optical assembly having at least one optical sensor configured to optically excite the defect center located within the semiconductor material and to detect a radiation emitted by the defect center depending on an optical excitation,
at least one microwave antenna configured to excite an electron spin state of the defect center located within the semiconductor material, and
at least one microwave drive and control circuit configured to drive the microwave antenna and the semiconductor material, wherein the defect center coincides with at least one of the zero magnetic field lines;
wherein an electron spin and an energy state of the defect center are allowed to be changed when excited by an electromagnetic energy.

2. The magnetic field measuring device according to claim 1, wherein the semiconductor material comprises at least one diamond having negatively charged nitrogen-vacancy defect centers in a structure of the semiconductor material.

3. The magnetic field measuring device as in according to claim 1, further comprising at least one compensation coil configured to generate at least a second magnetic field for adjusting a position of the zero magnetic field lines by moving the zero magnetic field lines generated by the transmitting coils.

4. The magnetic field measuring device according to claim 3, wherein the compensation coil is configured to generate the second magnetic field having a magnetic field gradient, and the magnetic field gradient eliminates a variation with a position of the first magnetic field generated by the transmitting coils around the zero magnetic field line.

5. The magnetic field measuring device according to claim 3, wherein a sine-shaped time-varying current is applied to the compensation coil to manipulate a magnetization of the magnetic particles.

6. The magnetic field measuring measurement device according to claim 5, wherein the coil drive and control circuit, the optical assembly, and the microwave drive and control circuit are configured to communicate in such a way as to exchange data with each other in order to apply signal waveforms necessary for a magnetic field measurement to the transmitting coils, the compensation coils, the defect centers, and the microwave antenna.

7. The magnetic field measuring measurement device according to claim 1, wherein the microwave drive and control circuit is configured to control the microwave antenna, wherein a microwave signal modulated at a frequency is emitted to the defect centers, wherein hyperfine electron spin resonances are excited at the frequency.

8. The magnetic field measuring device according to claim 1, wherein a plurality of transmitting coils are positioned at different positions around the sensing region to be measured so as to increase an intensity of the first magnetic field in the sensing region to be measured or to homogenize the first magnetic field in the sensing region to be measured.

9. The magnetic field measuring device according to claim 1, further comprising at least one permanent magnet arranged to separate directions of the defect centers.

10. A magnetic particle detection system, comprising the magnetic field measuring device according to claim 1.

11. A magnetic particle imaging system, comprising the magnetic field measuring device according to claim 9.

12. A magnetic particle relaxometer system, comprising the magnetic field measuring device according to claim 1.

13. The magnetic field measuring device according to claim 2, further comprising at least one compensation coil configured to generate a second magnetic field for adjusting a position of the zero magnetic field lines by moving the zero magnetic field lines generated by the transmitting coils.

14. The magnetic field measuring device according to claim 4, wherein a sine-shaped time-varying current is applied to the compensation coil to manipulate a magnetization of the magnetic particles.

15. The magnetic field measuring device according to claim 4, wherein the coil drive and control circuit, the optical assembly, and the microwave drive and control circuit are configured to communicate in such a way as to exchange data with each other in order to apply signal waveforms necessary for a magnetic field measurement to the transmitting coils, the compensation coils, the defect centers, and the microwave antenna.

16. The magnetic field measuring device according to claim 5, wherein the coil drive and control circuit, the optical assembly, and the microwave drive and control circuit are configured to communicate in such a way as to exchange data with each other in order to apply signal waveforms necessary for a magnetic field measurement to the transmitting coils, the compensation coils, the defect centers, and the microwave antenna.

17. The magnetic field measuring device according to claim 2, wherein the microwave drive and control circuit is configured to control the microwave antenna, wherein a microwave signal modulated at a frequency is emitted to the defect centers, wherein hyperfine electron spin resonances are excited at the frequency.

18. The magnetic field measuring device according to claim 3, wherein the microwave drive and control circuit is configured to control the microwave antenna, wherein a microwave signal modulated at a frequency is emitted to the defect centers, wherein hyperfine electron spin resonances are excited at the frequency.

19. The magnetic field measuring device according to claim 4, wherein the microwave drive and control circuit is configured to control the microwave antenna, wherein a microwave signal modulated at a frequency is emitted to the defect centers, wherein hyperfine electron spin resonances are excited at the frequency.

20. The magnetic field measuring device according to claim 5, wherein the microwave drive and control circuit is configured to control the microwave antenna, wherein a microwave signal modulated at a frequency is emitted to the defect centers, wherein hyperfine electron spin resonances are excited at the frequency.
